(19) United States
(12) Patent Application Publication
(10) Pub. No.: US 2003/0114803 A1
(43) Pub. Date: Jun. 19, 2003

(54) FLEXIBLE SHEATH FOR INTRODUCING A MEDICAL DEVICE INTO A DUCT

(76) Inventor: ALEXANDER LERNER, REHOVOT (IL)

Correspondence Address: FROMMER LAWRENCE & HAUG, 745 FIFTH AVENUE- 10TH FL., NEW YORK, NY, 10151, US

(21) Appl. No.: 09/473,197

(22) Filed: Dec. 14, 1999

(30) Foreign Application Priority Data
Jun. 30, 1998 (US) PCT/IL98/00306
Jul. 3, 1997 (IL) 121224
Jan. 19, 1998 (IL) 122985

(51) Int. Cl. A61M 5/00

(52) U.S. Cl. 604/276; 604/271

(57) ABSTRACT

Tubular, endless body, adapted to be introduced into, advanced along, and retracted from a duct, particularly of the human body, which comprises, when distended by inner fluid pressure, an outer surface, an inner channel, and rounded (toroidal) ends connecting the said outer surface to the surface of said inner channel, and which comprises portions of different diameter when in distended condition.

### FIELD OF THE INVENTION

The invention relates to devices for introducing medical apparatus and the like into a duct and advancing such apparatus along said duct, which is generally a part of the human body.

### BACKGROUND OF THE INVENTION

PCT Patent Application PCT/IL 97/00077, Publication Number WO 97/32515, discloses an endoscopic insertion device which comprises a cylindrical tubular sheath, having an outer surface, an axial channel and rounded ends, hereinafter called “toroidal”, connecting said outer surface to the inner surface of the sheath, which is the surface of the axial channel. Said sheath, which forms an endless, tubular body, is capable of rolling upon itself, so that different portions of its outer and inner surface proceed from the proximate to the distal end, or vice versa, and/or may slide along the duct into which the sheath is introduced. The sheath is maintained distended by filling it with gas or liquid under moderate pressure.

Such sheaths are useful for introducing medical devices into ducts of the body, particularly the human body. Certain medical devices, however, may be difficult or painful to introduce and activate. For instance, some endoscopes have a tubular structure that is stiff or rigid and the introduction of which is painful, and which further are not so constructed as to visualize dearly and completely the part of the duct surface which is of interest. Other medical devices are those for running an enema. These comprise a tube for introducing water, which tube is relatively stiff to permit its introduction, but bends and comes into contact with the intestine surface and is therefore difficult to use and painful Further, all those medical devices may be difficult to advance past obstacles, such as bends or convolutions, of a duct, such as an intestine.

U.S. Pat. No. 5,045,070 discloses an everting tube having an inner wall and an outer wall which together form a continuous elongated annular cross-section that is uniform over the length of the tube, said tube having an elongated open channel. A flexible introduction device is provided, the forward portion of which has a truncated conical section that can be inserted into a body cavity orifice, whereby the tube can be everted into the body cavity through said introduction device.

While the sheath described in said PCT Patent Application WO 97/32515 is extremely useful, it has been found that its general structure can be improved to increase its flexibility, to facilitate its advance along a duct into which it is introduced, particularly when the duct has a restricted section or bend or convolution or the like, and to increase its usefulness for various purposes, such as will be set forth hereinafter. The same is true with regard to U.S. Pat. No. 5,045,070, the improvement of which is only concerned with facilitating the introduction of an everting tube into a body cavity.

Reference will be made hereinafter to the use of the improved sheath according to the invention as “an introductory aid for medical apparatus”, but this application is intended to cover its structure regardless of the use which is made of it, and therefore the reference to medical uses should not be construed as a limitation The duct into which the sheath may be introduced may be a duct of the body of a living being other than a human being, or may be a duct of a structure or apparatus of any kind.

It is therefore an object of this invention to provide a device for introducing medical apparatus into a duct and advancing it along said duct, which has improved flexibility.

Another object of this invention is to provide such a device which can be introduced into a duct having sharp bends, and can be advanced therein passing through said sharp bends.

It is a further purpose of this invention to provide an apparatus and a method for facilitating the introduction of medical devices into ducts of the human body and/or their displacement and/or their activation therein.

It is a still further purpose of this invention to provide an apparatus and a method for improving the manipulation and use of medical devices to be introduced into ducts of the human body.

It is a still further purpose of this invention to provide means for eliminating or reducing the pain or discomfort connected with the use of medical devices,.such as stiff endoscopes or enema devices.

It is a still further purpose of this invention to provide means for facilitating the use of endoscopic observation devices by dilating the portion of the duct of the body to be observed.

It is a still further purpose of this invention to provide means for facilitating the use of endoscopic devices in ducts having restricted sections or other obstacles.

It is a still further purpose of this invention to provide an apparatus and a method for facilitating enemas.

Other purposes and advantages of this invention will appear as the description proceeds.

### SUMMARY OF THE INVENTION

As has been said, the device of this invention has in common to those described in the aforementioned PCT Patent Application WO 97/32515 the fact that it comprises an endless, tubular body, capable of rolling upon itself, or “sheath”, as it will be briefly called hereinafter, which in its distended condition has an outer surface, an inner channel, and rounded (toroidal) ends connecting the said outer surface to the surface of said inner channel. It has also in common with the sheath disclosed in the aforementioned application, the fact that it is kept in a distended condition by filing it preferably with a fluid under controlled pressure. The term “fluid”, as used herein, includes a single fluid —a liquid or a gas —or a combination of fluids, e.g., a liquid or liquids and a gas or gases.

Thus, the invention provides an aid for the introduction and use of medical devices into a duct of a mammal's body, preferably the human body, as well as a means for other uses such as the removal of foreign bodies or tissues from such a duct, which comprises a tubular sheath having an outer surface, an axial channel and rounded ends connecting said outer surface to the surface of said axial channel. In view of its structure and the method of its use, the sheath can roll upon itself, so to speak, so that different portions of its outer and inner surface proceed from the proximate to the distal end or vice versa, and, at other times or concurrently, can slide along the duct into which it is introduced. The combination of the sliding and rolling motions and the nature of the sheath, which is made of soft plastic film and reacts pneumatically, being filled with gas or liquid under moderate pressure, permits to introduce inspection or treatment devices, which are placed in the inner axial channel, into a duct of the human body, e.g. an intestine, and to displace them along said duct, with great ease and without pain, in spite of the contact of the sheath with the walls of the duct.

As has been said, the sheath, in use, is filled with a fluid under moderate pressure and therefore is in a distended condition. For this purpose, it is preferably provided with inlet means for a gas or liquid, generally air or water. Reference will be made hereinafter to air or to water, but this is not to be construed as a limitation. When in the distended condition, as has been said, the sheath has an outer surface and an axial channel, and in cross-section is annular in shape.

The sheath or endless, tubular body of this invention is characterized in that, in its distended condition, it has a greater flexibility, or a negative reduced resistance to bending, than the sheaths of the prior art. Such a greater flexibility is preferably achieved by providing the sheath with portions having different diameters, at least when the sheath is in its distended condition.

According to an embodiment of the invention, the sheath has a structure that may be called “chain-like”, inasmuch as it comprises successive portions of higher and lower resistance to bending. Since it is preferred that the endless, tubular body have an essentially circular cross-section at all points, the aforesaid chain-like structure is preferably achieved by providing an endless, tubular body having portions of different diameters, viz. larger diameter segments, which can be called “body segments” or “link segments”, intercalated by portions of smaller diameter, which can be called “necks” or “pivot joints”. Preferably, the segments having different diameters alternatingly follow one another, and in this case the sheath will be defined as an alternating diameter sheath or a sheath having an alternating diameter profile. The said definition should be construed as including therein sheaths in which the segments having different diameters alternatingly follow one another only along a portion of the length of the sheath; and everything that will be said about sheaths having an alternating diameter profile will apply to sheath that have such a shape only along a portion of their length. The geometric and configurational characteristics hereinafter mentioned are those of the sheath in its distended condition, unless otherwise specified. The diameter of the sheath's portions of largest diameter is determined by the use that it is intended to make of the sheath, mainly by the diameter of the duct into which it is to be introduced. Said largest diameter will also be called hereinafter “the nominal diameter”.

Alternating diameter sheaths, according to the invention, may be of various configurations. For examples, they may comprise: a) substantially cylindrical segments having the largest diameter (which is substantially the nominal diameter), substantially cylindrical segments of smaller diameter, and substantially conical segments, connecting said segments of largest diameter to said segments of smaller diameter; or b) convex portions, e.g. having approximately a sinusoidal profile, adjacent to one another or separated by ring-like portions having said minimum diameter. Also, the larger diameter portions or segments, on the one hand, and the smaller diameter portions or segments, on the other hand, may be all of substantially the same length along the sheath, which, in this case, may be called a regular, alternating diameter sheath; or said portions or segments may have variable lengths, in which case the sheath may be called an irregular, alternating diameter sheath. The said conical segments may have a substantial length and be generally V-shaped, or may be very short, in which case they may he called “grooves”.

In another form of the invention, a sheath that has increased flexibility or reduced resistance to bending and portions of different diameter is obtained not by a chain-like structure, but by undulating or corrugating its surface. In some cases the undulations or corrugations are helically shaped, so that the outer surface of the sheath is “screw-like” in shape. The peaks of the undulations or corrugations are the portions of nominal diameter.

In an embodiment of the invention, the necks or pivot joints are significantly shorter than the body or link segments. Preferably, in this case, the necks are circular grooves separating successive body segments. Such a sheath may be said to be “sausage-like” in shape.

According to another embodiment of the device, the portions of smaller diameter are constituted by segments having gradually decreased diameter from their end sections to their center section, viz. a V-shape, the sides of the V being straight or curved and the apex of the V constituting the actual pivot in correspondence to which the resistance to bending is at a minimum and the bending of the sheath is essentially concentrated.

This novel structure increases the flexibility of the sheath, or endless, tubular body, so that it can overcome obstacles more easily, can adapt itself to any bends or convolutions of the duct, and therefore can carry out all the functions and achieve all the results desired in such devices, in a more efficient manner and even under more difficult conditions. It has been found that said novel structure permits the device to progress along ducts of such a conformation that would impede the progress of prior art devices.

When the sheath, or endless, tubular body, has helically shaped undulations or corrugations, it can also be advanced into a duct not only by rolling upon itself, in the manner described in the aforementioned PCT Patent Application WO 97/32515, but also by rotating as a screw rotates, and because of the helical configuration of the corrugations or undulations of its outer surface, such rotation in one direction will advance it into and along the duct (“screw it in”) and a rotation in the opposite direction will retract it along and from the duct (“screw it out”).

In all forms of the invention, the sheath can advance into a duct by rolling upon itself, in the manner described in the aforementioned PCT Patent Application WO 97/32515, e.g. by pushing it forward, viz. applying pressure in the direction from its proximate to its distal end, by means of a pushing rod or an instrument that is inserted into the inner channel defined by the sheath in its distended condition. In the same manner, it can be caused to regress along a duct or be withdrawn from a duct. The fact that the sheath has an alternating diameter surface, which comprises portions of smaller diameter, or valleys or grooves, does not interfere with its rolling motion. The sheath can also be advanced or withdrawn by causing it to slide along a duct, whenever this is required for any reason, and/or by screwing it.

Hereinafter the sheaths will be illustrated as of limited length, in order to simplify the figures. However, the length of the sheath is determined, of course, by the distance of the point that it is intended to reach in the duct from the inlet of said duct.

The sheath or tubular, endless body of the invention can be used to introduce and maneuver endoscopes or other instruments, to introduce or remove liquids from the duct and the parts of the body with which the duct communicates, and to carry out any other function that may required. It can be said that the sheath or endless, tubular body according to the invention, is particularly useful for introducing optical devices, for draining fluids or performing cleaning actions by means of fluids and for reaching far into a duct, even in the presence of obstructions, convolutions and the like, to carry out the aforesaid operations.

In a form of the invention, particularly designed to remove from a duct foreign bodies, such as fragments of tissue resulting from a surgical operation, stones, or in general any solid body or particle, the device according to the invention comprises, in addition to a main or outer sheath as hereinbefore defined, an inner sheath inserted in the axial channel of the outer sheath and axially displaceable with respect to it, which is advanced beyond the distal end of the outer sheath to seize the foreign body and is then withdraw into the outer sheath, carrying the foreign body with it.

In another form of the invention, means are provided for sensing any obstacle to the progress of the sheath into the duct, whether caused by convolutions of the duct or by other factors, by sensing the resulting increase of pressure within the sheath. This makes possible to carry out any particular action that may be required by the situation.

The invention further comprises a method of introducing the sheath, with the medical apparatus which it is intended to carry with it, into the opening of a duct, as well as means for carrying out said method, all as will be described hereinafter.

In a further embodiment of the invention, which is of particular usefulness for introducing an endoscopic device for visual purposes, a portion of the sheath, which is beyond the distal end of the endoscope when this latter is connected to the sheath, viz. is in a more distal position than said distal end, is of larger diameter and/or more yieldable to pressure than the other parts of the sheath. Endoscopes normally have a rigid or stiff stem and an operative, foremost end attached to the front of the stem. When the endoscope is in use, its operative, foremost end is also its distal end, so that, when reference is made to the operation and use of the introduction aid according to the invention, “foremost end” and “distal end” can be regarded as having the same meaning. At the beginning of the use of the sheath, and when gas or liquid under pressure is admitted into this latter, said larger and/or more yieldable portion is within the sheath and is part of its axial channel. But after the sheath has been introduced into a duct and when said portion reaches the distal end thereof, it swells outwardly more than the remaining parts of the sheath and extends the duct into which it has been introduced, to facilitate visualizing.

The terms “distal end” and “proximate end”, when used hereinafter without specification, refer to the configuration of the sheath at the beginning of its use, when it has not been introduced into a duct, while the ends that are the distal and proximate one when the sheath is in use, within a duct, may be called hereinafter “the actual distal end” and “the actual proximate end”, respectively.

A method of using with a medical device a sheath having a portion of larger diameter than the other portions thereof, particularly when the medical is an endoscope having an operative, foremost end —in the case of an endoscope, the visualizing end —and a rigid or stiff stem to which said end is attached, comprises connecting said foremost end to the sheath at or near to the proximate end of the aforesaid larger sheath portion, introducing said foremost end into the axial channel of the sheath at the proximate end of this latter, whereby said larger diameter portion becomes the proximate part of the surface of said axial channel and is subject to compressive pressure, bringing the sheath to its distended condition by admitting fluid into it, placing the distal end of the sheath into the orifice of a duct of the human body, and advancing the sheath with the medical device into the duct by causing the sheath to roll upon itself, e.g. by pushing on the stem of said device, whereby said stem of said device and said larger portion of said sheath progress along the axial channel of the sheath, until said sheath portion, with the foremost end of the medical device, has reached the desired, actual distal position. This latter, of course, is determined by the length of the sheath, which, in this method, must correspond to the distance of said desired, actual distal position from the orifice of the duct. At this point, said larger diameter portion constitutes the actual distal rounded end of the sheath, and not being constrained any longer by other portions of the sheath and being subject to expanding pressure, swells outwardly and distends the wall of the duct. Thereafter, the sheath is withdrawn, causing it to slide over the wall of the duct, without any change in its configuration, e.g. by pulling it from its actual proximate end, and the medical device is operated, visualizing the duct, the successive portions of which become successively distended, during the sliding retraction of the sheath. By “larger portion of the sheath” is meant a portion of greater diameter than the remaining portions or a portion that is more yieldable to pressure and can acquire or increase its greater diameter only when subjected to inner pressure. Therefore the term “larger” should always be understood as meaning “of larger and/or more yieldable to pressure”.

The invention further comprises a method of producing an alternating diameter sheath according to the invention, which comprises: 1 —providing a cylindrical tube, 2 —imparting to it the desired alternating diameter profile, in particular by the application of inner pressure and heat, 3 —folding it into itself until both ends thereof are in proximity to one another, whereby two segments of the tube are positioned one inside the other and a cylindrical pocket is defined between said two segments of the tube, 4 —optionally, if required, introducing into the said cylindrical pocket a desired fluid, and 5 —connecting said tube ends to one another to form a tubular, endless body.

### BRIEF DESCRIPTION OF THE DRAWINGS

FIG. 1 is a longitudinal cross-section, partly in lateral view, of a sheath or endless, tubular body according to an embodiment of the invention;

FIG. 2 is a transverse cross-section of the sheath of FIG. 1, taken along plane II-II of FIG. 1;

FIG. 3 is a partial lateral view of a sheath or endless, tubular body according to another embodiment of the invention;

FIG. 4 is a schematic cross-section of a two-layer toroidal tube;

FIG. 5 is a schematic cross-section illustrating the use of a sheath according to the embodiment of FIG. 1 for visualizing a duct;

FIG. 6 schematically illustrates the introduction of the sheath according to an embodiment of the invention into a duct having sharp bends;

FIG. 7 schematically illustrates means for measuring the resistance encountered by a sheath according to the invention when advanced along a duct;

FIGS. 8 (a), (b) and (c) illustrate the use of a sheath according to the invention for withdrawing foreign bodies from a duct;

FIG. 9 schematically illustrates an embodiment of a method of making an alternating diameter sheath according to the invention;

FIG. 10 schematically illustrates a double-toroidal tube;

FIG. 11 is a schematic, axial cross-section of a further embodiment of the invention, showing the actual distal portion of the sheath and of an endoscope when fully introduced into a duct of the human body;

FIG. 12 is a transverse cross-section of the same along plane XI-XI;

FIGS. 13 to 15 schematically illustrates the phases of a method of using the embodiment of FIGS. 11 and 12;

FIGS. 16 to 18 schematically illustrate the phases involved in meeting an obstacle with a device according to an embodiment of the invention;

FIGS. 19 to 21 illustrate in schematic axial cross-section an enema sheath, shown in the phases of its insertion into a duct;

FIG. 22 is a transverse cross-section of FIG. 21 on plane XXII-XXII;

FIG. 23 is a schematic illustration of a liquid pressure control apparatus;

FIG. 24 illustrates a situation in which a sheath meets with a obstacle in a duct; and

FIG. 25 shows diagrams of the liquid pressure variation in the situation of FIG. 24.

### DETAILED DESCRIPTION OF PREFERRED EMBODIMENTS

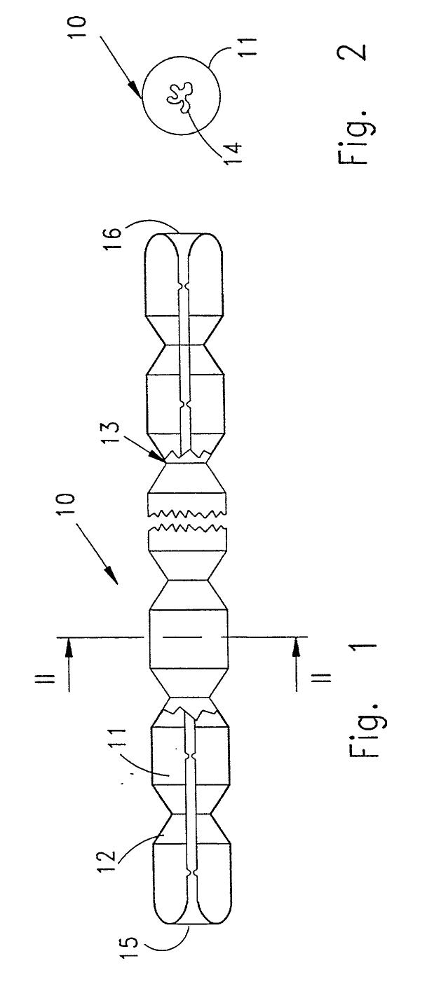

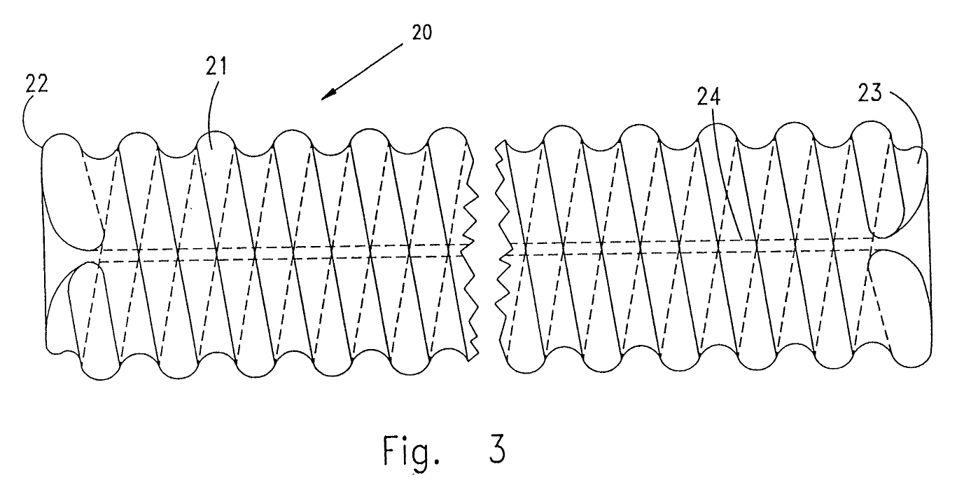

Referring now to FIG. 1, a sheath or tubular, endless body according to an embodiment of the invention, is generally indicated at 10. FIG. 1 shows the sheath as partly in view, and broken off, to show only the segments adjacent its distal end 15 and its proximate end 16. As seen in the figure, the sheath comprises alternating cylindrical segments 11 and V-shaped segments 12 connecting segments 11. The diameter of segments 11 is the nominal —viz. the largest —diameter. The smallest diameter is that of the vertex of the V-shaped segments, as indicated at 13. The inner channel 14 has an irregular cross-section, as shown in FIG. 2, which is a consequence of the fact that the inner channel is not inflated.

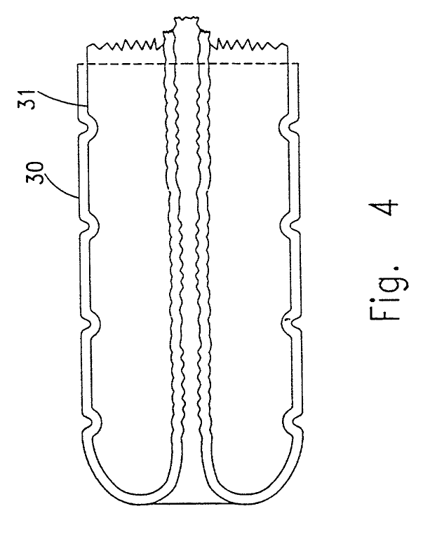

FIG. 3 shows another, second, embodiment of a sheath according to the invention, illustrated in lateral view and as broken off in the middle. The sheath 20 of this embodiment is undulated or corrugated on its surface, the undulations or corrugation having a helical configuration and being indicated at 21. The lands of the helix have the nominal diameter. 22 is the distal, toroidal end of the sheath and 23 is its proximate, toroidal end. 24 is the inner channel. It is seen in the cross-section of FIG. 3 that the profile of the surface of the sheath, in longitudinal cross-section, is similar to a sinusoidal configuration.

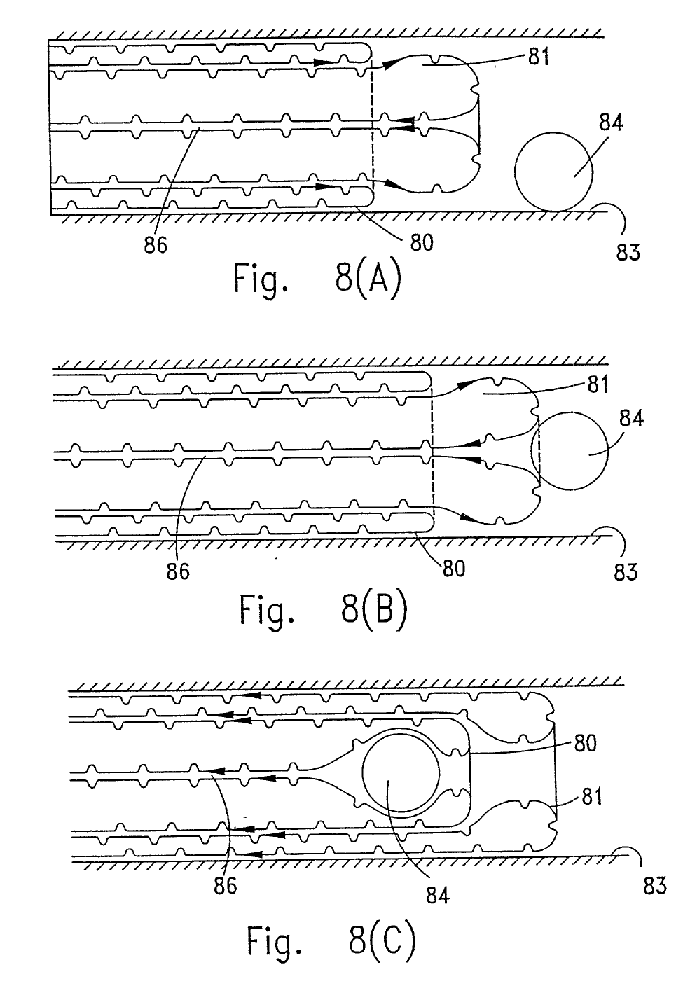

FIG. 4 illustrates another embodiment of the invention, in which the sheath is constituted by two component sheaths 30 and 31 inserted the one into the other. It has been found that the creation of sections of different diameters may create zones of decreased wall thickness, particularly, thinner zones may be generated, by the stretching of the sheath walls, at the junctions between adjacent sections of different diameters, so that failure may occur at said zones. For examples, said thinner zones may have a wall thickness that is about one half the wall thickness of the uniform portions of the sheath. On the other hand, it has been found that it is not desirable to strengthen said zones by increasing the wall thickness all over the sheath. This problem can be overcome by using a double or composite sheath, viz. two equally shaped component sheaths inserted the one into the other. For instance, if the method of making the sheath illustrated in FIGS. 9 (a) and (b) is used, two concentric tubes 90 will be placed into die 91 and will be shaped concurrently. It has been found that the mechanical properties of such double sheaths, and particularly their combination of mechanical strength and flexibility, are extremely good.

In the embodiments described, all the larger diameter segments have the same configuration and the same length, and all the smaller diameter segments have the same configuration and the same length, but, as has been said, this is not necessary, and the length of the larger segments as well as that of the smaller segments could be varied along the sheath, to produce an irregular sheath. This should be understood, though it will not be repeated, with respect to any embodiment of the invention.

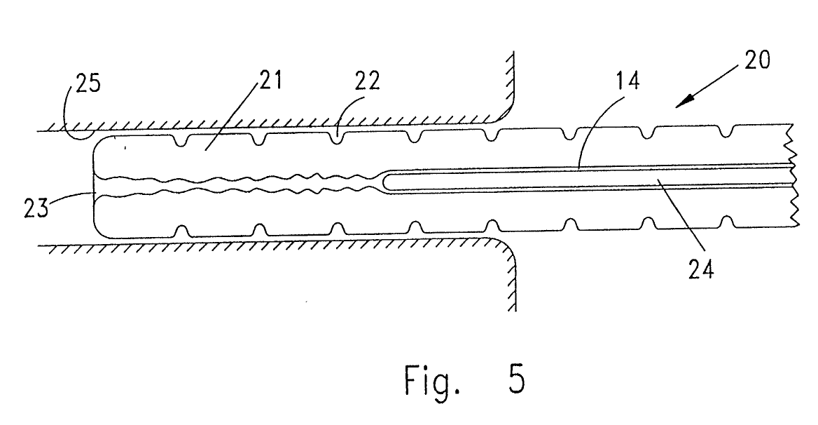

FIG. 5 schematically illustrates a possible use of the sheath according to the invention, which is shown, by way of example only, as having the configuration illustrated in FIG. 1, but which could have any other configuration as long as it is an alternating diameter sheath. In FIG. 5, the 20 has been introduced into a duct 25 and has proceeded, by rolling upon itself, as far as desired into the duct. Numeral 24 illustrates an endoscope which is inserted into inner channel 23 of sheath 20 and pushed forward, to cause sheath 20 to roll upon itself and progress along duct 25, until its final position (not illustrated) is reached, wherein the endoscope projects from the sheath and visualizes the duct.

All the aforesaid embodiments of the invention have flexibility that is sharply improved over that of prior art devices. This feature is of considerable value in every case, but becomes essential, and even indispensable, when the device must advance along a sharply bent duct.

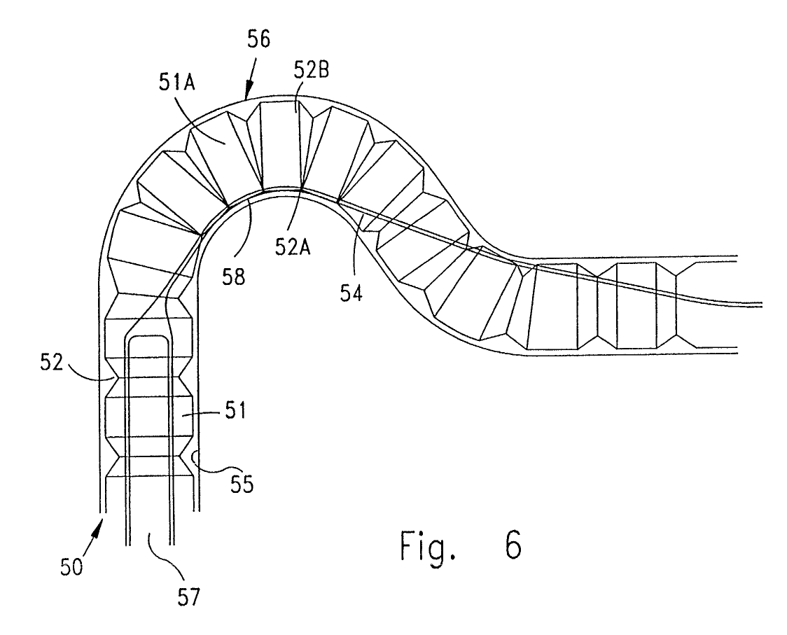

Such a case is illustrated in FIG. 6, in which a sheath 50 is shown, by way of example, as having a configuration similar to that of the embodiment of FIGS. 1 and 2.

Duct 55, within which sheath 50 has been introduced and must progress, is sharply bent duct 56. A device 57, which may be a visualizing device, such as e.g. an endoscope or a TV-camera providing with a connecting cable, is inserted into sheath 50, but for it to progress beyond bend 56, the sheath must firstly progress beyond the same. Sheath 50 has cylindrical segments 51 connected by v-shaped segments 52. It is seen that, when sheath 50 reaches bend 56, segments 51 swing with respect to one another, about the vertices of segments 52, as particularly visible in the relative position of cylindrical segments 51A and 51B and v-shaped segment 52A, thereby facilitating the bending of the sheath and its progress beyond bend 56 deeper into the duct. While the sheath bends, both its segments 51 and its segments 52 may become somewhat deformed, in view of their flexibility, their portions adjacent the inside of the bent contracting somewhat and their portions adjacent the outside of the bent stretching somewhat. As a result, inner channel 54 surprisingly shifts to one side of the sheath, as shown at 58. for advancing along a sharply bent duct, as shown at 58. The device 57 or the cable which connects a device such as a TV-camera to visualizing apparatus, must also be flexible in order to progress beyond the bend.

It is to be noted that it is not always necessary that the sheath of the invention should have an alternating diameter profile, or a chain structure constituted by a succession of links separated by pivot joints, or, in particular, should comprise cylindrical segments and V-shaped segments connecting the cylindrical segments, along all its length, as in the embodiments illustrated. It is sufficient, in many cases, that it have such a structure along a portion of its length, where lower bending resistance is required. For instance, if an endoscope is inserted into the sheath, it is sufficient that the distal portion of the sheath, beyond the distal end of the endoscope, should have such a structure, for the sheath to progress beyond a bend of a duct, as shown in FIG. 6. Said distal portion may have a length, for instance, of about 3 to 20 cm. Once the distal portion of the sheath has so progressed, the remaining portion of the sheath will so progress as well, even if it has a constant diameter cylindrical shape, and entrain with it the endoscope or other device.

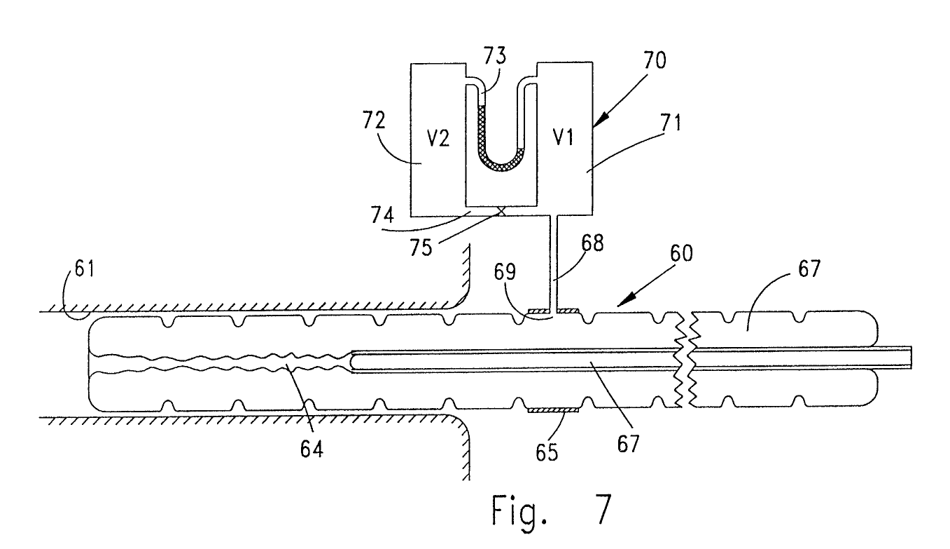

FIG. 7 illustrates a method and a device for sensing obstacles to the progress of the sheath according to the invention into a duct, due to convolutions of the duct or other obstacles, so that the operator may take whatever steps are required.

A sheath 60, which is illustrated in cross-section as having the configuration of FIGS. 1 and 2, but could have any other configuration according to the invention, is provided, and into its inner channel 64 a device, such as an endoscope, schematically indicated at 67, is introduced. Sheath 60 associated with a rigid or at any rate sufficiently consistent sleeve 65, which is connected by a tube 68 to a differential manometer 70, hereinafter described. Sheath 60 is perforated at 69 in correspondence to tube 68. At the beginning of the operation, sheath 60 is retracted so that its distal end slightly protrudes from sleeve 65. Thereafter, the sheath is advanced into the duct in the ordinary manner, while the sleeve remains motionless and close to the opening of duct 61, as indicated in the drawing.

The differential manometer 70 comprises two chambers, 71 and 72, connected by a U-tube 73, which contains, e.g., mercury. The chambers 71 and 72 are filled with gas, e.g., air. Said chambers are connected at the bottom by a tube 74, which comprises a capillary, or at least very narrow, opening 75 (viz., a “jet”), created in any suitable manner, e.g. by locally restricting the tube or by providing in it a partition perforated to form said opening.

If sheath 60 meets with a resistance in its progress along the duct, such as a convolution of the same or an obstacle, and the operator urges it forward, the pressure inside the sheath increases and increased pressure is transmitted through tube 68 to the chamber 71 of the differential manometer. Therefore gas will flow from chamber 71 to chamber 72 to balance the pressure in the two chambers, but since it must flow through the aforesaid passage 75, but it will do so slowly, because of the very small cross-section of passage 75, so that, for a certain length of time, the pressure in chamber 71 will be higher than in chamber 72, and this will be evidenced by a displacement of the mercury in U-tube 73.

FIGS. 8(a), 8(b) and 8(c) illustrate an embodiment of the invention and a method of using it for extracting foreign bodies, of any origin, from a duct. This embodiment comprises an outer sheath 80 and an inner sheath 81. Outer sheath 80 can be advanced along duct 83 in the manner of all the embodiments of this invention, and inner sheath 81 can be advanced along and inside outer sheath 80, in the same manner. However, inner sheet 81 moves in a “backward” direction relative to outer sheet 80, because its outer surface moves in the same direction as the inner surface of sheet 80.

In the position of FIG. 8(a), the device has progressed into duct 83 to the vicinity of a foreign body 84 of any kind of origin. In FIG. 8(b), the device has been further advanced so that the toroidal, distal end of inner sheath 81 has come into contact with the foreign body 84. If outer sheath 80, carrying with it inner sheath 81, is further advanced, inner sheath 81 grips body 84 by its backward motion and “swallows” it, and this latter penetrates through the distal end into its inner channel 862. Thereafter, inner sheath 81 is retracted into outer sheath 80 until it reaches the position of FIG. 8(c), wherein it is entirely within the inner channel 86 of sheath 80. The pressure exerted by the surface of channel 86 on the distal end of sheath 81 causes the same to close over and around foreign body 84, as seen in FIG. 8(c). Thereafter, both sheaths can be withdrawn from duct 83.

Of course, actuating means, such as rigid or semi-rigid tubes, must be provided, one for advancing the outer sheath 80 into the duct, and the other one for advancing the inner sheath 81 within the outer sheath 80. These tubes and other advancing means are not shown in the drawings, for simplicity of illustration, as their provision is obvious and involves no difficulty.

FIG. 9 schematically illustrates an embodiment of a method of making a sheath according to the invention. A cylindrical tube 90 of thermoplastic material is inserted into a die 91 having an inner surface 92, the diameter of which varies along its length. In this embodiment, surface 92 has the shape illustrated in FIG. 1, constituted by cylindrical segments and V-shaped segments connecting the cylindrical segments, but, in general, it will have the shape that matches the desired outer shape of the sheath to be made, e.g., the shape shown in FIG. 3 or 4. Die 91 is heated in any suitable way. FIG. 9 shows a die immersed in a liquid 93, contained in a container 94, and heated by electric resistance means schematically indicated at 95, but any other heating means could be adopted. A pressure is created within tube 90 by any means, as schematically indicated at 96. Under the influence of heat and pressure, tube 90 swells, as symbolically indicated by broken line 97, as far as surface 92 will permit, and adopt the shape of said surface. Thereafter, the tube is removed from the die and, once cooled, it is folded on itself, and the two adjacent ends thereof are sealed, as described for a cylindrical tube in the aforesaid application WO 97/32515. A portion of enlarged diameter can be obtained by welding two pipe segments of different diameter together or by distending to some extent a portion of a single pipe segment.

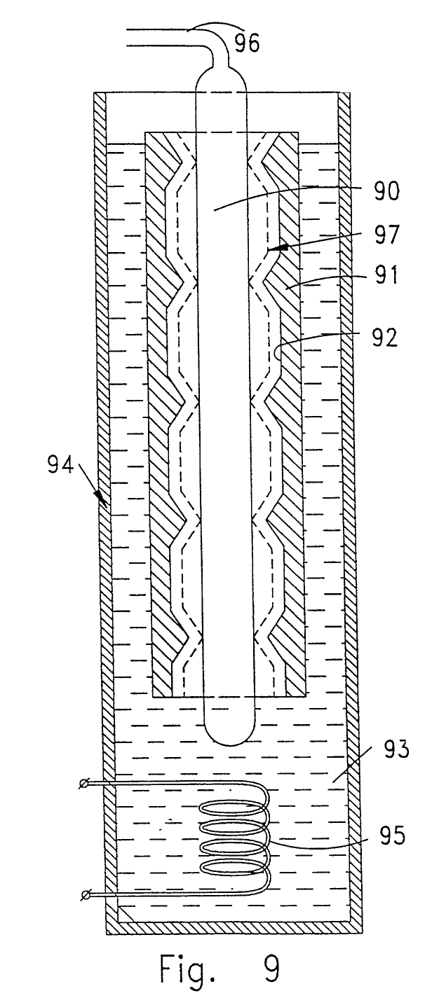

Another embodiment of the invention, comprising a double sheath, is shown in FIG. 10. Inside an alternating diameter tube 100 is located a cylindrical tube 101. A pressure P₁ is maintained inside tube 100, as shown al 102, and a pressure P₂ is maintained inside tube 101, as shown al 103, the two pressures being controlled independently. Such a double-toroidal sheath provides substantial advantages in controlling the process of advancing the sheath along a duct.

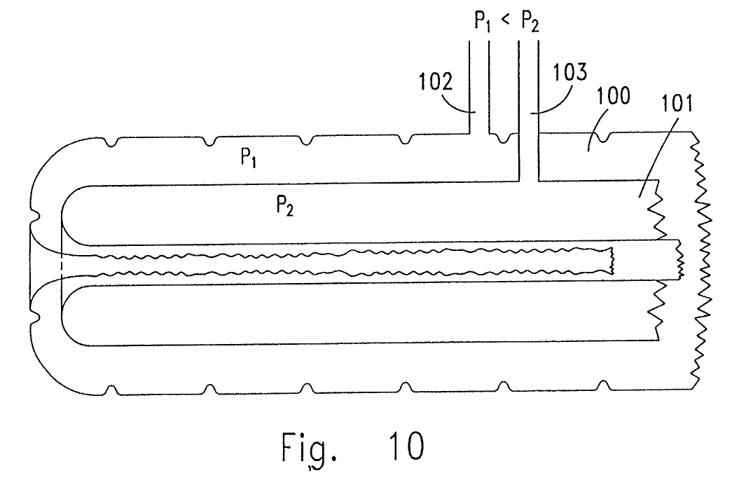

In the embodiment of FIGS. 11 and 12, numeral 120 designates the inner surface of a duct of the human body, into which an endoscopic device has been introduced. The sheath according to the invention is indicated at 121 and has an outer surface 122 and an axial channel 123. An endoscope 124, having a cylindrical stem and an optical head with a transparent cover 127 is inserted into channel 123. The cover 127 is connected to the inner surface of sheath 121 at 125. In this position, the actual distal portion of sheath 121, indicated at 126, is swollen out; so that it correspondingly swells the duct into which the device is introduced to afford a better view of the duct surface indicated at 128. The broken lines at 129 indicate the field of view of the endoscope itself.

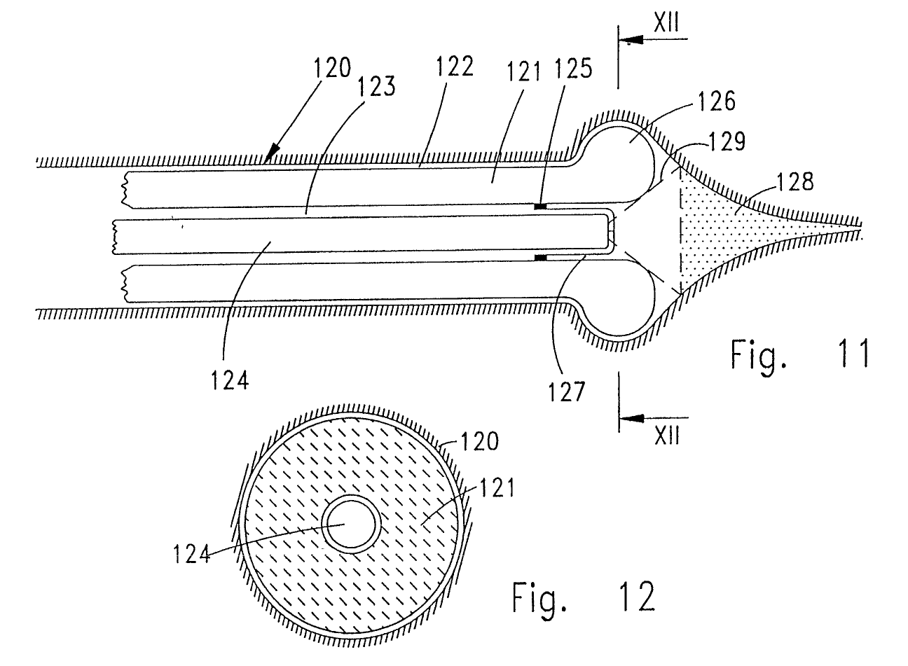

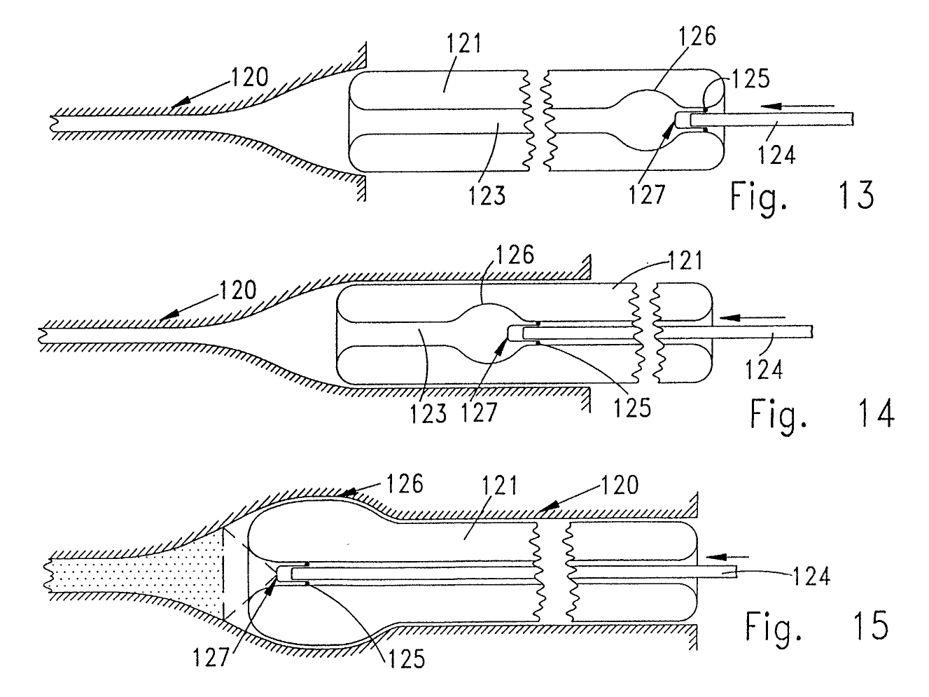

FIGS. 13 to 15 schematically illustrate one way of using the embodiment of FIGS. 11 and 12. The sheath 121, the length of which may be in the order of 1 meter or even more, is shown as broken off for the necessity of illustration and endoscope 124 is also shown as broken off. At the beginning of the operation (FIG. 13), the endoscope 124 is entirely outside the sheath 121 close to the transparent cover 127. The larger portion 126 of sheath 121 is inside the channel 123. The distal end of sheath 121 is then introduced into duct 120, and the endoscope is pushed forward. This causes the sheath to roll upon itself and it proceeds into duct 120. The beginning of this stage is illustrated in FIG. 14. As the introduction proceeds by pushing on the stem of endoscope 124, the device reaches the position of FIG. 15, in which it is fully introduced, and the larger portion 126 is no longer in axial channel 123 and swells out. At this point, the endoscope is activated and begins to visualize the inside of duct 120. The sheath 121 with the endoscope is gradually pulled back, sliding along duct 120 and as it so slides, the swollen portion 126 swells successive portions of the duct 120, as illustrated at 120′ in FIG. 15, which shows an intermediate position of the phase of the operation in which the sheath 121 with the endoscope has retreated from the actual distal position, but not yet reached the orifice of duct 120.

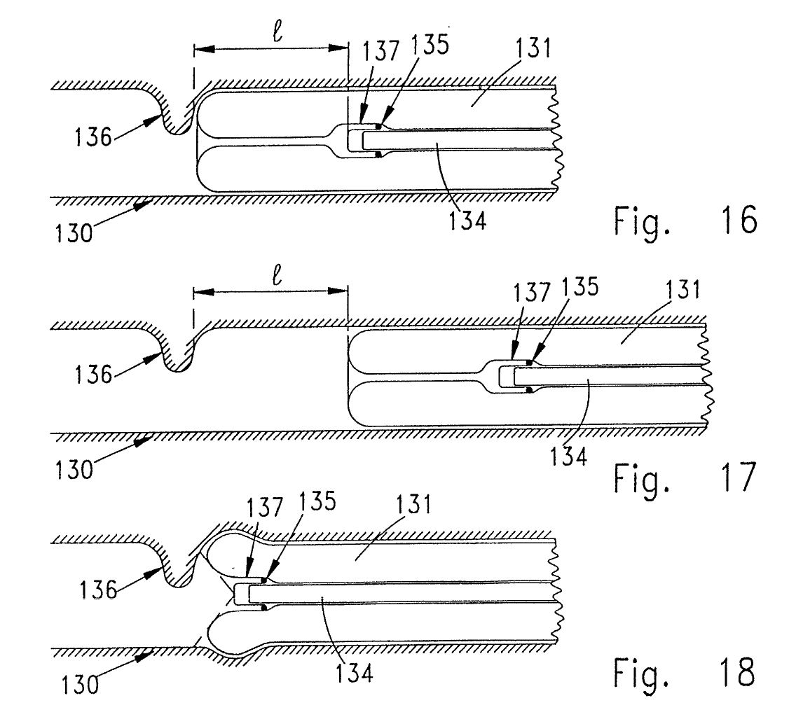

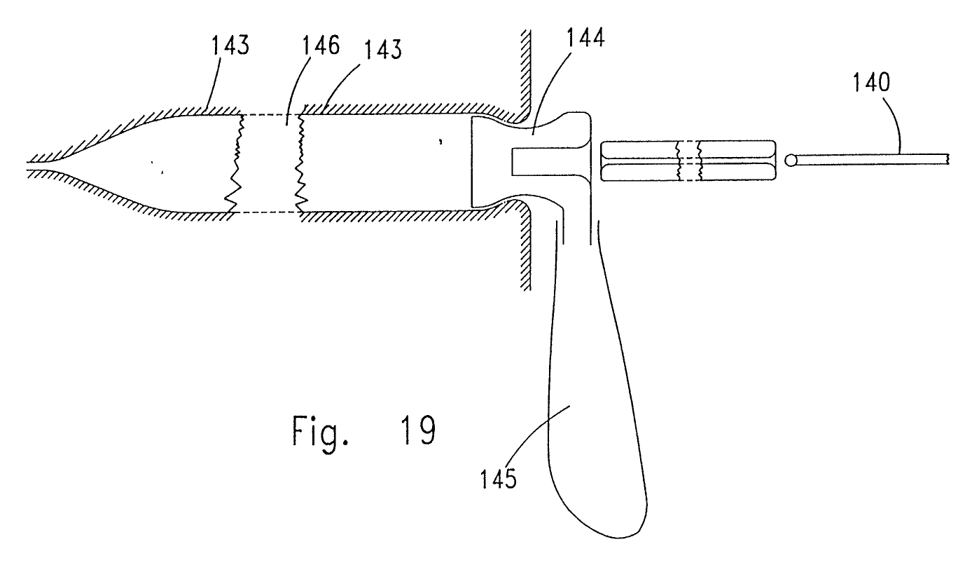

By rolling forward and back and by sliding forward and pulling back of the sheath 121, the transparent cover 127, with the head of the endoscope inside, may be located at any point of the duct in an open position, shown in FIG. 15. For instance, let us presume that during the introduction of the sheath into the duct, an obstacle 136 (FIG. 16) was met, when the cover 137 was located at a distance “1” from the front of the sheath, and the operator wants to see what is the obstacle. In such a case, it is possible to pull the sheath until the distance between the obstacle 136 and the front of the sheath will reach the value of “1”, as is shown in FIG. 17, then to roll in the endoscope 134 until it comes to the position shown in FIG. 18, when the obstacle 136 may be observed and evaluated.

In a device according to the invention, particularly suitable for running an enema, the sheath is supplemented with a drainage container for receiving the water which flows between the sheath and the duct into which it is introduced, which drainage container has an inlet overlapping, with some clearance, the proximate end (which, in this embodiment, is also the actual proximate end) of the sheath. A pipe, with a sprayer, is inserted into the axial channel of the sheath from its distal end (which, in this embodiment, is also its actual distal end), with the sprayer remaining outside said distal end, and is connected with a water source at its proximate end. The water issuing from the sprayer passes between the sheath and the inner surface of the intestine, then into the clearance between the sheath and the drainage container inlet and collects in said container.

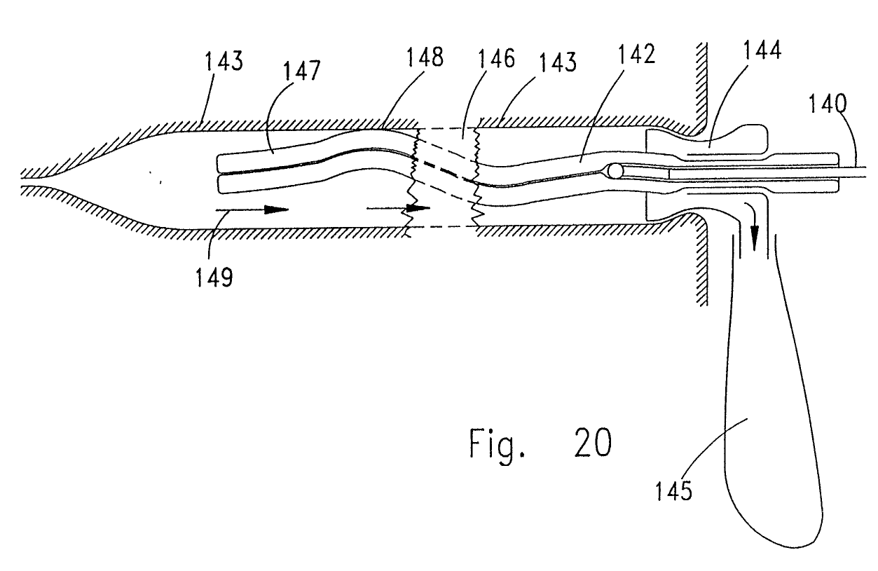

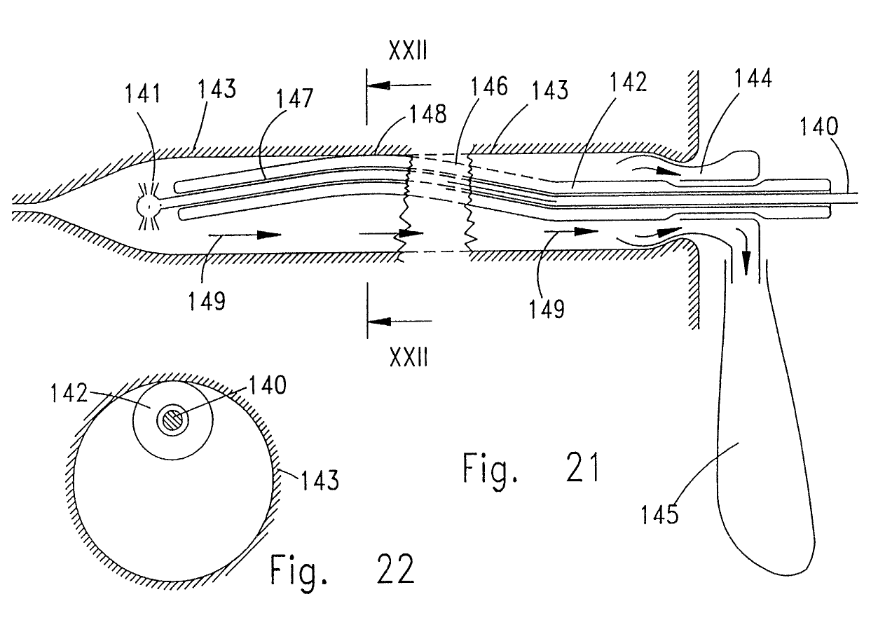

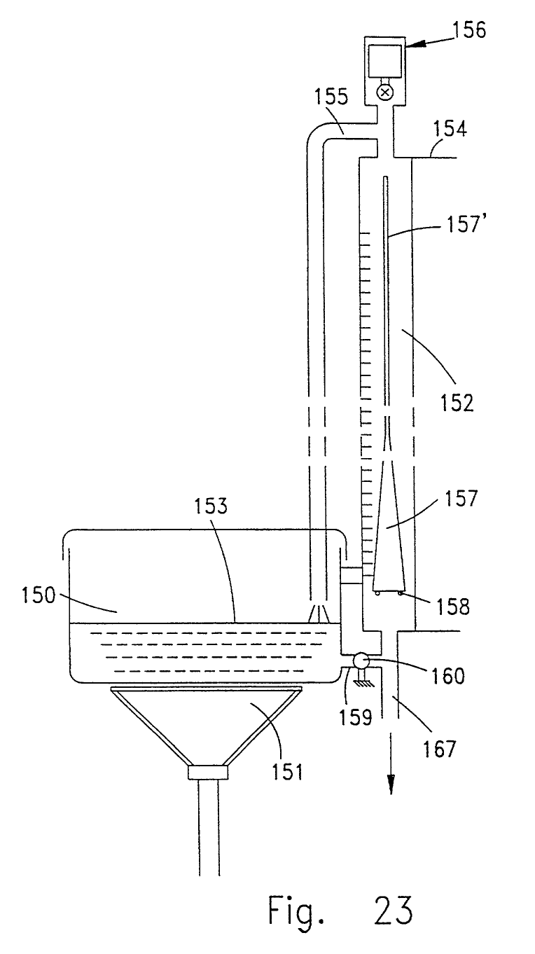

Such a device is illustrated (as broken off) in FIGS. 19 to 22. It comprises a pipe 140, connected to a water supply and terminating in a spray head 141, is introduced into a duct by a sheath 142, which is similar to that of FIGS. 11 and 12, but of a diameter substantially smaller than that of the intestine 143 into which the device is to be introduced, and has an outer surface 146 and an axial channel 147. The terminal 144 constitutes the inlet of a drainage container 145, a clearance existing between sleeve 144 and sheath surface 146.

At the beginning of the process of using this enema sheath, the spray head 141 of the water pipe 140 is introduced into the sheath 142 from its proximate end (FIG. 19). Then the sheath is advanced in the duct 143 by rolling upon itself (FIG. 20). At the final stage of introduction, shown in FIG. 21, the spray head 141 comes out of the sheath 142 at its distal end and the water supply can be switched on. When the washing of the intestine is over, the device can be pulled off from the duct.

The water introduced from tube 140 flows back, as indicated by arrows 149, to and into the space between the sheath 142 and the duct 143, enters terminal 144 and collects in drainage container 145.

In an embodiment of the invention, in which the fluid used to fill the sheath is a liquid, preferably water, means are preferably provided for feeding the liquid to the sheath, monitoring its pressure and controlling the changes in pressure that may derive e-g. from various situations arising in the use of the sheath, or from variations of the cross-section and the configuration of the duct into which its is introduced, or from the displacements of the medical device in the axial channel of the sheath, or from any combinations of these factors. Said means preferably comprise a tank which contains a supply of the liquid, optionally under pressure, a pressure tube, conduit means placing said tank and said tube into hydraulic communication with one another and with the inside of the sheath, and adjusting valve means for controlling the flow of liquid between said tank and said conduit means. The pressure tube preferably consists of a vertical or essentially vertical tube wherein the level of the liquid may freely oscillate, extending to a height above that of said tank, more preferably provided with an overflow to said tank and to means for varying the cross-section available to the liquid at different levels.

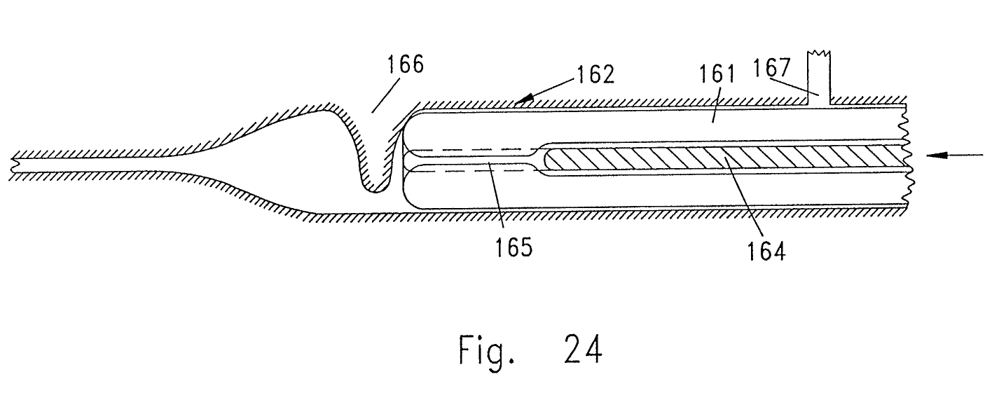

FIG. 23 illustrates such an apparatus. The apparatus comprises a supply tank 150, which may be sealed and under pressure, and generally has a volume of a few liters, for instance, 2 liters. The supply tank 150 is conveniently placed on a fixed support 151. Numeral 152 designates a pressure tube, which is a vertical tube, generally having a diameter of a few centimeters, e.g. 3-4 cm, and a height in the order of 1 meter or the like, which extends vertically, or essentially vertically, from the level of plane 153, which is an intermediate level of the supply tank, to an upper level indicated at 154. The level of the liquid in said tube can freely oscillate and is only constrained by an overflow tube 155, which returns any liquid substantially exceeding the level 154, to the supply tank. Desirably, lighting means generally indicated at 156 are provided, to permit an observer to see what the level of the liquid in the tube is. An insert 157 is provided inside the tube 152. The insert has a cross-section which varies at different levels, and preferably is largest at the bottom, where the insert rests on supports 158 and decreases from the bottom up, so that the insert ends in a rod-like portion 157′.

Tube 152 is connected at its bottom to a conduit 167, only the beginning of which is shown, and which communicates with the inside of the sheath according to any embodiment of the invention in which a liquid is used. Supply tank 150 also communicates near its bottom through a connection 159 with conduit 167. An adjusting valve 160 is provided in said connection 159, so as to control, depending on its lumen, the flow of liquid in either direction through connection 159.

When the sheath is to be filled by liquid, valve 160 is opened to any desired degree and liquid flows to the sheath. If tube 152 was originally empty, the liquid flows into the tube as well, and reaches the same level as the level of liquid in the tank 150. During the operation of the device, the volume of the sheath can vary for various reasons. Firstly, if a medical device is inserted into the axial channel of the sheath, said device has a certain size and will dilate the axial channel, tending to increase the pressure within the sheath, decreasing the volume available to the liquid within said sheath. Secondly, the volume of the duct occupied by the sheath, as it advances into it, may vary, depending upon the diameter of the duct and the variations thereof along its length. Other factors may produce increased pressure. When the pressure increases, the liquid, since it is incompressible, flows back to the supply tank and to the pressure tube. If the adjusting valve is completely open, so that the resistance in connection 159 between the supply tank and the pressure tube is negligible, the liquid will flow into the pressure tank and raise the level therein only slightly, causing a small increase in pressure in the tank, and will rise in the pressure tube only to the extent that corresponds to the increased pressure above the liquid in the tank. If the valve is partly closed and poses significant resistance to the flow of liquid, the level in tube 152 will increase in order to overcome not only the increase of pressure in the tank, but also the resistance of the valve. The ratio of the liquid level in the pressure tube will be faster at the beginning, when the pressure is low, because the cross-section of the tube available to it is smaller due to the presence of the insert 157, and will become slower as the pressure increases. The operation of the adjusting valve 160 will permit to control the level; variation rate in the pressure tube, and in general the operation of the apparatus of FIG. 23.

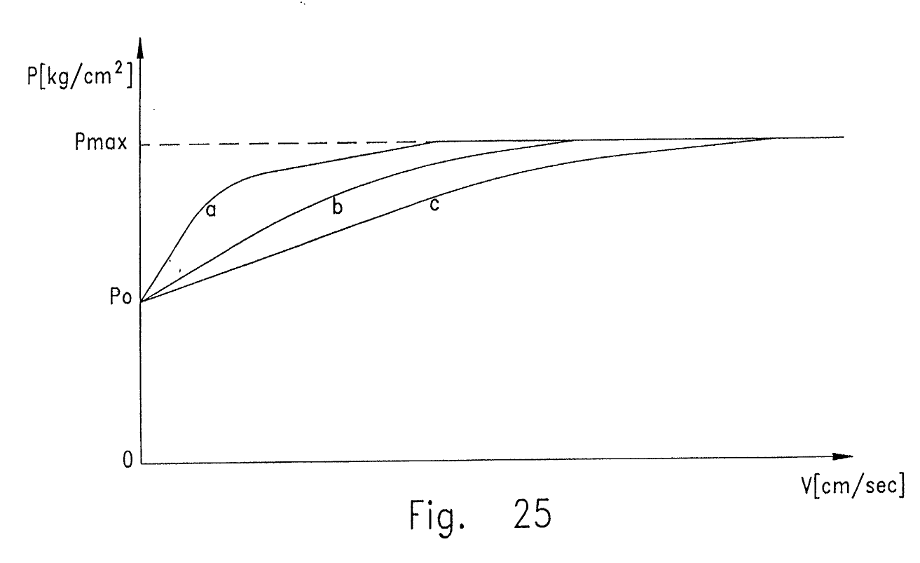

A situation in which the apparatus of FIG. 23 is adapted to control an increase of pressure is schematically illustrated in FIG. 24. It shows a sheath 161 introduced into a duct 162. A medical device 164 of substantial diameter is introduced into axial channel 165 of sheath 161. A conduit 167, which is assumed to be the continuation of conduit 167 of FIG. 23, places the sheath in communication with the apparatus of FIG. 23. As the sheath with the medical device progresses along duct 162, it meets, in this case, with an obstacle 166, viz. a growth of any type, which so extensively obstructs duct 162 that sheath 161 cannot progress any longer. The space available within the sheath to the liquid will have decreased and therefore the pressure will have increased, and this will be reflected in variations of the liquid level in pressure tube 152 of FIG. 23.

How these variations depend on the speed with which the medical device 164 is advanced, in this case or in a similar one, and on the lumen of adjusting valve 160, is illustrated in FIG. 25, which shows the action of the liquid pressure as a function of the speed (indicated by V). The medical device is advanced after the sheath has met with the obstruction 166, the rate at which the pressure increases also depends on the lumen of the adjusting valve 160 and three different curves, a, b and c, are illustrated, relating to the lumen of said valve which decreases from that of curve a to that of curve c. In the diagram of FIG. 25, the initial pressure within the sheath is assumed to be 0.1 kg/cm² and reaches a maximum value of 0.2 kg/cm².

The sheath according to the invention is made of flexible plastic film, preferably of a thickness from 5 to 25 μm. Preferred plastic materials of said film are e.g. polyethylene and polyurethane. It can be made by skilled persons in any suitable way, for instance, by folding a length of a thin flexible tube upon itself, so that one section of it becomes inserted into another section and the two ends of the tube become adjacent, folding one of said ends over the other, and connecting them by welding or adhesively or in any other suitable manner. Aforesaid PCT Patent Application WO 97/32515 illustrates the preparation of a similar sheath. The length of the sheath, as well as its diameter and the size and position of the larger portion, if one is provided, depend on the particular application, by the size of the duct into which it is to be introduced, and by the distal from the orifice thereof of the actual distal position that it is desired to reach. All these factors are easily taken into account by skilled persons.

A method of using the device of the invention with a medical device, e.g. an endoscope, having an operative foremost end - in the case of an endoscope, its visualizing end - and a rigid or stiff stem to which said end is attached, which method is particularly useful when the duct into which the sheath is to be introduced has an obstacle, such as a restricted section or bend or convolution, comprises connecting said operative foremost end to the sheath at or near the proximate end thereof, admitting fluid into the sheath until has been brought to its distended condition by it, rolling the sheath upon itself by pushing on the stem of the medical device until the foremost end of this latter is near the distal end of the sheath, e.g. by pushing on the stem of the medical device, and sliding the sheath without rolling into the duct as far as possible, while the medical device is operated, e.g. the endoscope visualizes. If an obstacle, such as an obstruction or a bend or convolution of the duct, is met, the medical device is pulled back by a short distance, the sheath correspondingly rolls upon itself so that its actual distal portion is free of the foremost end of the medical device and becomes pliable, pushing forward the sheath until its actual distal end has passed the obstacle, then rolling the sheath upon itself, e.g. by pushing on the stem of the medical device, until its foremost end has reached once again the actual distal end of the sheath amid has passed through the obstruction, and resuming the sliding progression of the sheath into the duct until the final, actual distal, position is reached or another obstruction is met. Once the operation of the medical device had terminated, the sheath can be withdrawn by causing it to roll upon itself, e.g. by pulling on the stem of said device.

While embodiments of the invention have been described by way of illustration, it will be understood that the invention can be carried out by persons skilled in the art with many modifications, variations and adaptations, without departing from its spirit or exceeding the scope of the claims.

1. Tubular, endless body, adapted to be introduced into, advanced along, and retracted from a duct, particularly of the human body, which comprises, when distended by inner fluid pressure, an outer surface, an inner channel (14), and rounded toroidal ends (15,16-22,24) connecting the said outer surface to the surface of said inner channel, characterized in that it comprises portions (11,12) of different diameter when in distended condition.

2. Tubular, endless body according to claim 1, comprising portions of reduced resistance to bending.

3. Tubular, endless body according to claim 1, having, at least along a portion of its length, a chain structure constituted by succession of links (11), separated by pivot joints (13).

4. Tubular, endless body according to claim 3, wherein the links are cylindrical segments (51) and the joints are grooves (52).

5. Tubular, endless body according to claim 3, wherein the links are cylindrical segments (51) and the joints are V-shaped segments (52).

6. Tubular, endless body according to claim 1, having a helical outer surface (21).

7. Tubular, endless body according to claim 1, having an essentially circular cross-section at all points, said cross-section having different diameters at different points.

8. Tubular, endless body according to claim 1, having portions of different wall thickness.

9. Tubular, endless body according to claim 1, comprising a medical device (124) inserted into its inner channel, and wherein a portion (126) of the sheath (121), which is beyond the distal end of said device, is of larger diameter and/or more yieldable to pressure than the other parts of the sheath.

10. Tubular, endless body according to claim 1, having an alternating diameter profile.

11. Device for removing foreign bodies from a duct, characterized in that it comprises, an outer tubular, endless body (80) according to claim 1 and an inner tubular, endless body (81) according to claim 1, inserted in the axial channel of said outer body and axially displaceable with respect to it.

12. Device for sensing any obstacle to the progress of a tubular, endless body into a duct, characterized in that it comprises means for sensing an increase of pressure within the sheath.

13. Device according to claim 12, wherein the means for sensing an increase of pressure within the sheath comprise a sleeve (65) for receiving therein an endless, tubular body (60), an orifice (69) in said sleeve registering with an orifice in said body, a differential manometer (70) having two chambers (71,72), and tube means (68) connecting said orifice of said sleeve with one of said chambers of said manometer.

14. Method of using an introduction aid according to claim 9, which comprises connecting the distal end of the medical device (124) to the sheath (121) at or near to the beginning of the portion of larger diameter and/or more yieldable to pressure of the sheath, introducing said distal end into the axial channel (123) of the sheath at the proximate end of this latter, bringing the sheath to a distended condition by admitting a gas or liquid into it, placing the distal end of the sheath into the orifice of a duct (120) of the human body, advancing the sheath with the medical device into the duct by pushing on the stem of said device while the sheath rolls upon itself, whereby said stem and said portion of larger diameter and/or more yieldable to pressure progress along said axial channel until they reach a desired actual distal position, whereby said portion of larger diameter and/or more yieldable to pressure swells outwardly and distends the wall of the duct, starting to operate the medical device, and withdrawing the sheath by causing it to slide over the wall of the duct, while continuing to operate the medical device.

15. Method for running an enema, which comprises providing an enema apparatus having a water adduction tube (140) and a sprayer (141) and an introduction aid according to claim 1, further comprising a drainage container (145) having an inlet (144) overlapping, with a clearance, the proximate of the sheath (142), which method comprises inserting the enema apparatus into the axial channel of the sheath from the distal end thereof, with said sprayer protruding from said distal end, feeding water to said water adduction tube and collecting water into said drainage container.

16. Method of producing an alternating diameter sheath according to claim 1, which comprises: providing a cylindrical tube, imparting to it the desired alternating diameter profile, folding it into itself until both ends thereof are in proximity to one another, whereby two segments of the tube are positioned one inside the other and a cylindrical pocket is defined between said two segments of the tube, and connecting said tube ends to one another to form a tubular, endless body.

17. Method according to claim 16, wherein the desired alternating diameter profile is imparted to the tube by the application of pressure and heat.

18. Composite sheath, comprising two endless, tubular bodies according to any one of claims 1 to 10, inserted into one another.

19. Composite sheath, comprising two endless, tubular bodies, inserted into one another, the outer one being a tubular body according to any one of claims 1 to 10, and the inner one being a cylindrical tubular body.
